(19) United States
(12) Patent Application Publication
(10) Pub. No.: US 2023/0405035 A1
(43) Pub. Date: Dec. 21, 2023

(54) MONTBRETIN A FUNCTIONAL FOOD

(71) Applicant: THE UNIVERSITY OF BRITISH COLUMBIA, Vancouver (CA)

(72) Inventors: John-paul Heale, Vancouver (CA); Spence Stott Skene MacDonald, Vancouver (CA); Kyle Brian Robinson, Vancouver (CA)

(73) Assignee: THE UNIVERSITY OF BRITISH COLUMBIA, Vancouver, BC (CA)

(21) Appl. No.: 18/251,510

(22) PCT Filed: Oct. 21, 2021
(86) PCT No.: PCT/CA2021/051485

§ 371 Date: May 2, 2023

Related U.S. Application Data
(60) Provisional application No. 63/109,140, filed on Nov. 3, 2020.

(51) Int. Cl. A61K 31/7048; A21D 2/14; A61K 9/00

(52) U.S. Cl. CPC A61K 31/7048; A61K 9/0056; A21D 2/14

(57) ABSTRACT

Functional food comprising montbretin A and use of the functional food for limiting polysaccharide processing in a human subject and for treating conditions treatable by inhibiting pancreatic a-amylase.

### CROSS REFERENCE TO RELATED APPLICATION

This application claims the benefit of U.S. Provisional Patent Application Ser. No. 63/109,140 filed on 3 Nov. 2020, entitled “MONTBRETIN A FUNCTIONAL FOOD”.

### TECHNICAL FIELD

A functional food product comprising montbretin A and use of the food product for limiting polysaccharide processing in a warm-blooded mammal.

### BACKGROUND OF THE INVENTION

Montbretin A has been shown to be effective in the inhibition of pancreatic α-amylase. Pancreatic α-amylase is an enzyme in the digestive system, catalyzing the initial step in the hydrolysis of starch, a principal source of glucose in the diet. It has been demonstrated that the activity of human pancreatic α-amylase (HPA) in the small intestine correlates to post-prandial glucose levels, the control of which is an important factor in diabetes and obesity. Thus, modulation of α-amylase activity through the therapeutic use of inhibitors is of considerable medical relevance.

Despite the advancements in the use of montbretin A for inhibiting pancreatic α-amylase and controlling starch digestion, a need exists for improved compositions and methods for effectively delivering montbretin A. The present invention seeks to fulfill this need and provides further related advantages.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 compares high performance liquid chromatography (HPLC) analysis Montbretin A (MbA) bread extracts: 2 mM MbA standard; MbA− bread extract; MbA+ bread extract. In the chromatographs MbA peak appears at about 17 min.

FIG. 2 shows human pancreatic α-amylase (HPA) kinetics with the colorimetric substrate, 2-chloro-4-nitrophenyl-β-D-maltotrioside (CNPG3).

FIG. 3 shows HPA inhibition with MbA bread extract. Inhibition assay performed with bread extract diluted ×1000 or ×10000.

FIG. 4 summarizes the results of a MbA bread IC₅₀ assay: plot displays one of three assays per sample (see FIG. 8 for all samples). MbA standard converted to nM using the conversion: 1 mM=6.15 g/mL. Error bars indicate 95% confidence range.

FIG. 5 compares HPLC analysis of IC₅₀ MbA bread extract samples: MbA*, MbA+, MbA−, and MbA standard.

FIG. 6 tabulates the quantification of MbA by HPLC. MbA mass conversion done by integrating the MbA peak at about 17 min of FIG. 5 and converting to MbA grams using the standard curve shown in FIG. 7.

FIG. 7 shows the HPLC MbA standard curve. Conversion equation:

$$MbA mas ( μ g ) = \frac{y - 252.3}{145.7} .$$

FIGS. 8A-8C compare MbA bread IC₅₀ assay results: MbA− (8A), MbA+ (8B), and MbA* (8C), relative to MbA standard. MbA standard converted to nM using the conversion: 1 mM=6.15 g/mL. Error bars indicate 95% confidence range.

### DETAILED DESCRIPTION OF THE INVENTION

The present invention is directed functional foods that include montbretin A (MbA). Montbretin A is a complex plant flavonol glycoside that is a potent and specific inhibitor of human pancreatic α-amylase with potential as a therapeutic for diabetes and obesity. As an inhibitor pancreatic α-amylase, montbretin is useful for controlling starch digestion and thereby useful in managing postprandial glycemia in pre-diabetic or diabetic subjects and/or for management of obesity in any subject.

Montbretin A is a complex flavonol glycoside having the structure:

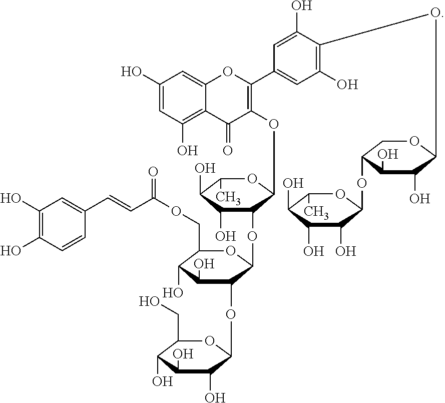

The inventors have surprising found that montbretin A, with a multiplicity of labile glycosidic linkages, is stable to cooking conditions (e.g., elevated temperature in the presence of complex mixtures of ingredients) to provide functional foods that include montbretin A. These montbretin A functional foods are cooked food products, such as baked or fried food products, that stably maintain montbretin A and its activity for inhibiting human pancreatic α-amylase. The montbretin A functional foods of the invention can be used to effectively deliver montbretin A to pre-diabetic or diabetic subjects and/or for management of obesity in a warm-blooded mammal (e.g., human, dog, cat) comprising montbretin A subject.

In one aspect, the invention provides a cooked food product for limiting polysaccharide processing in a warm-blooded mammal (e.g., human, dog, cat) comprising montbretin A.

As used herein, the term “cooked food product” is a food product that is heated at a temperature of at least 80° C. during its preparation prior to serving. Representative cooked food products are heated at a temperature from about 80 to about 150° C. In certain embodiments, the cooked food product is a food product that is prepared by heating to a temperature of about 100° C. for a period of time sufficient to provide the cooked product.

In certain embodiments, the cooked food product is a carbohydrate-containing food is a bread, a pasta, a dessert (pastries, custards, and puddings), or other foods made from dough.

In certain embodiments, the cooked food product is a baked food. In certain of these embodiments, the cooked food product is a baked food product that is prepared by baking a dough containing montbretin A, wherein the dough has a pH from about 4.5 to about 7.0.

As noted above, the cooked food product comprising montbretin A is useful for limiting polysaccharide processing in a human. As used herein, the term “polysaccharide” refers to a polymeric carbohydrate composed of monosaccharide units linked together by glycosidic units. Examples of polysaccharides include starch, which is a polymeric carbohydrate consisting of glucose units. Starch is produced by most green plants as energy storage and it is the most common carbohydrate in human diets and is contained in large amounts in staple foods such as potatoes, wheat, maize (corn), and rice. In the context of the present invention, in certain embodiments, polysaccharides include oligosaccharides and carbohydrates that include at least two simple sugars (e.g., glucose).

As used herein, “limiting polysaccharide processing in a human” refers to limiting the metabolic breakdown of polysaccharide in a human subject after the consumption of a polysaccharide-containing food by the human subject. The cooked food product of the invention is effective for limiting polysaccharide processing in the human subject. Representative polysaccharide-containing foods include baked foods such as breads, and pastries, as well as other polysaccharide-containing foods such as pastas and desserts (custards and puddings).

The cooked food product of Claim 1, wherein the montbretin A derivative is as described in U.S. Pat. No. 8,431,541, expressly incorporated herein by reference in its entirety.

In certain embodiments, the cooked food product includes montbretin A in an amount from about 0.1 to about 1.0 milligram per gram of the cooked food product.

In another aspect, the invention provides the use of montbretin A as an ingredient in a cooked food product for human consumption for limiting polysaccharide processing in a human.

In one embodiment, the invention provides a method for limiting polysaccharide processing in a human subject, comprising providing an effective amount of a cooked food product as described herein for consumption by a human subject in need thereof. In certain of these embodiments, the effective amount of the cooked food product is from about 0.1 gram to about 2.0 grams baked food product per kg human subject.

In another embodiment, the invention provides a method of managing postprandial glycemia in a human subject in need thereof, wherein said subject is pre-diabetic, has diabetes, or is obese, the method comprising providing an effective amount of a cooked food product as described herein for consumption by a human subject to manage said postprandial glycemia.

In a further embodiment, the invention provides a method of inhibiting mammalian α-amylase in a human in need of α-amylase inhibition, the method comprising administering an effective amount of a baked food product as described herein to inhibit said mammalian α-amylase. In certain embodiments, the α-amylase is pancreatic α-amylase or salivary α-amylase.

In another embodiment, the invention provides a method for treating diabetes in a human subject, comprising administering an effective amount of a cooked food product as described herein for consumption by a human subject for the treatment of diabetes.

The following is a description of the preparation and properties of a representative MbA food of the invention: bread containing MbA.

MbA Baking in Bread Assay

MbA was added as a primary ingredient to a standard bread recipe and to evaluate the integrity of the additive after baking. To this end MbA was added (45 mg) as part of the raw dough (225 g) and separated into 25 g half-servings after kneading. After baking to an internal temperature of 100° C., the samples were extracted with methanol and subject to HPLC and kinetic analysis. In parallel, control samples containing no MbA (MbA−) or MbA added after baking (MbA*) were assessed in an analogous manner. HPLC traces revealed 84% of MbA to be intact relative to control. Kinetic analysis was performed by measuring the inhibition of human pancreatic α-amylase (HPA) catalysis, extract samples (MbA+) had IC₅₀ of 323.9 μg/mL corresponding to 83% of the control inhibition activity. Overall, MbA was found to remain intact following standard bread production processes and retains greater than 80% of the activity of the unbaked controls (MbA*).

Materials and Methods

Baking

Two approximately 225 g batches of bread were prepared, one containing MbA the other with no MbA. For each batch:
1. 5 mL of yeast was activated by mixing with 62.5 mL 43.3° C. water and ¼ tsp sucrose then incubated on the bench top for 10 min.
2. ½ cup of all-purpose flour was mixed with ½ cup whole-wheat flour and ⅛ tsp NaCl (and 45 mg* MbA for the MbA+ batch only). *The target MbA concentration was 10 mg per serving of bread:

$$\frac{225 g of bread}{50 g of bread / serving} \times 0.01 g of MBA / serving = 0.045 g of MbA$$

3. The flour mixture was combined with the activated yeast mixture and 25 mL room temperature water then stirred until a dough formed.
4. Dough kneaded for 10 min by hand.
5. Dough was rested at room temperature for 30 min.
6. Dough was cut into 9 approximately 25 g mini-loafs, formed into a balls and rested in a muffin tin for another 30 min.
7. Baked for about 30 min or until the internal temperature reached 100° C. (internal probe thermometer was used to continuously monitor internal temperatures).
8. Mini loafs were cooled to room temperature before freezing at −20° C. until needed for testing.

Methanol Extraction
1. 1 MbA+ and 1 MbA− mini-loafs were quartered, weighted and placed in a 50 mL Falcon tube.
2. 5 mL of 100% methanol per 1 g bread was added to each segment.
3. The quartered segments were muddled with a glass stirring rod and placed in a 40° C. incubator for 2 h rotating at 350 rpm.
4. Stored at −20° C. until needed.
5. Methanol was separated by centrifugation then filtered through a 7 mL filter column.
6. Filtrate was evaporated in a speed vac until dry.
7. Dried material was resuspended in water to a final concentration of 6.15 g (of quartered loaf) per mL.
8. Stored at −20° C. until needed.

HPLC analysis

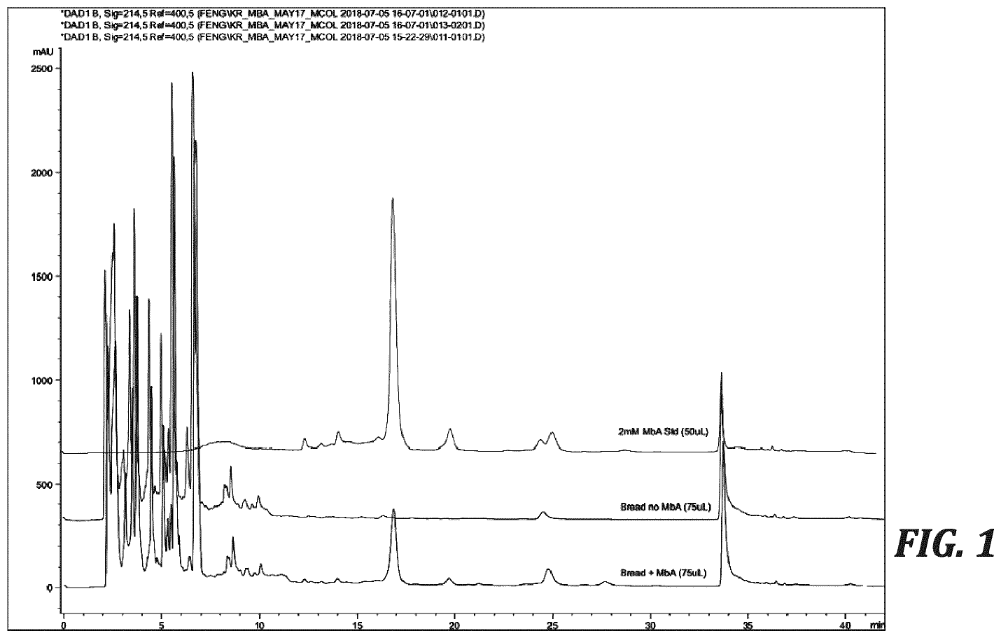

The HPLC analysis is shown in FIG. 1.
1. Aqueous extracts were injected on HPLC and separated over semi-preparative C18 column. Gradient elution from water (95%) to acetonitrile (95%) over 40 min afforded separation of sample. Data was visualized at 214 nm and 288 nm.
2. MbA standard was injected as control following above specifications.

HPA Inhibition Assay

MbA inhibits the activity of human pancreatic α-amylase (HPA). To determine whether and to what extent the prepared bread extract inhibits HPA, enzymatic assays were performed with the colorimetric substrate 2-chloro-4-nitrophenyl-β-D-maltotrioside (CNPG3).

HPA Activity Confirmation

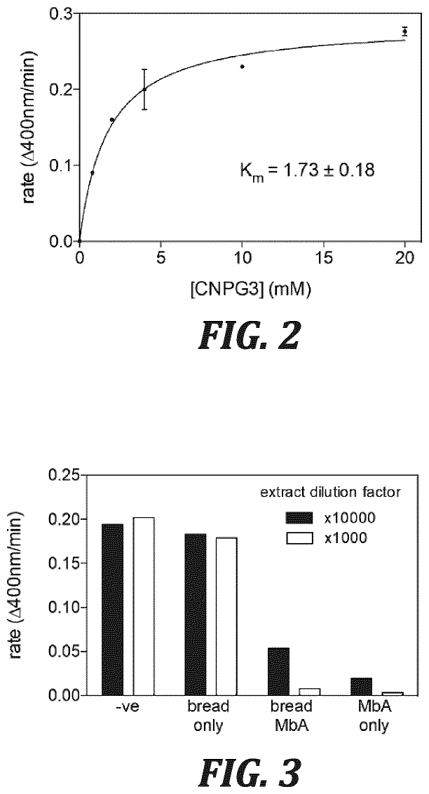

HPA inhibition activity of the bread prepared as described above was determined by monitoring CNPG3 concentration. See FIGS. 2 and 3.
1. 20 μL of 200, 100, 40, 20 or 8 mM CNPG3 were combined with 170 μL buffer (50 mM phosphate pH 7.0, 100 mM NaCl) in quartz cuvettes and placed in a CARYXXX UV/Vis spectrophotometer.
2. To each of the five cuvettes, 10 μL of HPA solution was rapidly added to the 190 μL buffered substrate solution.
3. Spectrophotometer was set to continuously measure absorbance at 400 nm over 5 min.
4. Repeat twice (n=3).
5. Rates were calculated from t=1 to t=3 (Δ400 nm/min), plotted against CNPG3 concentration and fit with the Michaelis-Menten equation.

Bread Extract HPA Inhibition

Bread extract HPA inhibition is summarized in FIG. 3.
1. 20 μL of 40 mM CNPG was combined with 150 μL buffer and 20 μL MbA− or MbA+ bread extract (either ×100 or ×1000 diluted with water). 2 additional controls were included: instead of bread extract, water (20 μL) or MbA (either 10 μM or 1 μM) was added.
2. To each cuvette, 10 μL of HPA solution was rapidly added to the 190 μL buffered substrate mixture.
3. Spectrophotometer was set to continuously measure absorbance at 400 nm over 5 min.
4. Rates were calculated from t=1 to t=3 (Δ400 nm/min), plotted on a bar graph.

MbA IC50

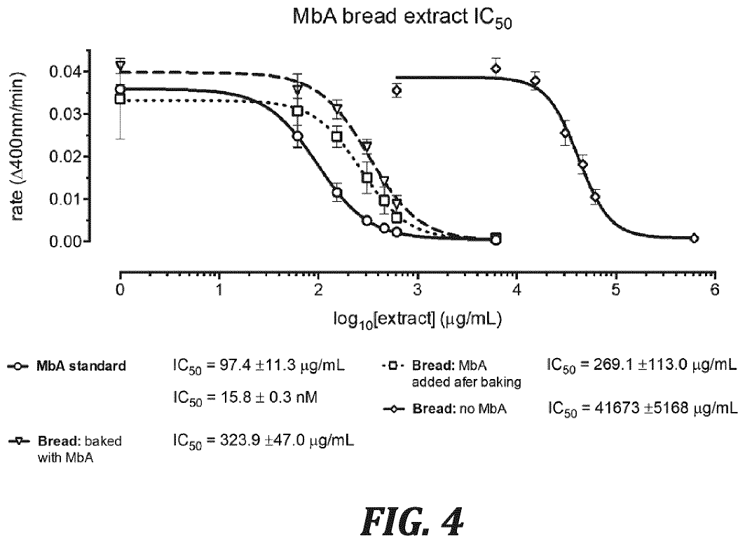

MbA IC₅₀ values for HPA inhibition were determined and are summarized in FIG. 4.

Three sets of bread extractions were done to measure IC₅₀:
A. Bread baked with MbA (MbA+)
B. Bread baked without MbA (MbA−)
C. Bread baked without MbA, but spiked with 0.2 mg/g MbA immediately prior to methanol extraction (MbA*)
   Methanol extraction was performed as above on the three mini-loafs. For the MbA* sample, MbA was added (as part of the 5 mL methanol/g of bread) to a final concentration of 0.2 mg/g of bread from a stock of 30 mg/mL MbA (in 100% methanol). IC₅₀s were measured using the HPA/CNPG3 assay described above:
1. For each of the 3 mini-loafs, 3 extracts were done each on a quartered segment, totaling 9 extracts. For each of those extracts 3 technical replicate IC₅₀ assays were performed. Additionally, IC₅₀ was measured for purified MbA. The maximum theoretical concentration of MbA in the 6.15 g/mL extracts (assuming 100% recovery from extraction) is 1 mM. Therefore, a sample of 1 mM MbA in 100% methanol was prepared and measured as a positive control.
2. For MbA+ and MbA* samples the 6.15 g/mL extracts and 1 mM MbA control were diluted ×100, ×1000, ×1333, ×2000, ×4000, ×10000 and ×100000. For the MbA− samples the 6.15 g/mL extracts were diluted ×1, ×10, ×13.3, ×20, ×40, ×100 and ×1000.
3. Measurements were performed in a 96-well microtiter plate with a Bio-tek plate reader with a final concentration of 4 mM CNPG3 at 30° C.
4. 20 μL of 40 mM CNPG was combined with 150 μL buffer, 20 μL of bread extract or MbA control dilution series and 10 μL HPA.
5. Absorbance at 400 nm was measured every 1 min after adding HPA for a total of 10 min. Rates were calculated from 5 to 10 min then plotted to determine IC₅₀ values.

HPLC Analysis of MbA for MbA Breads

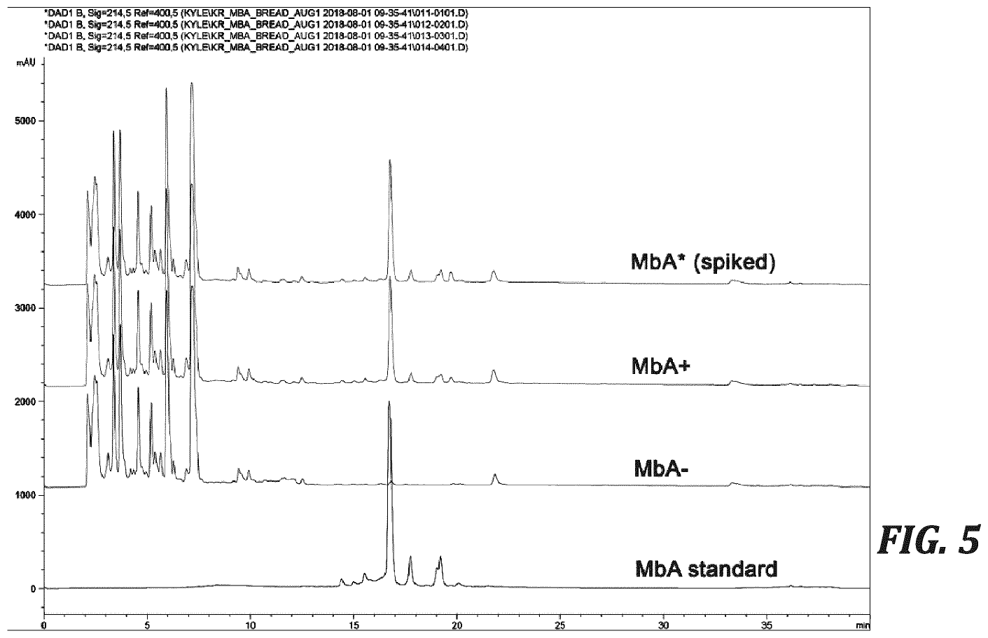

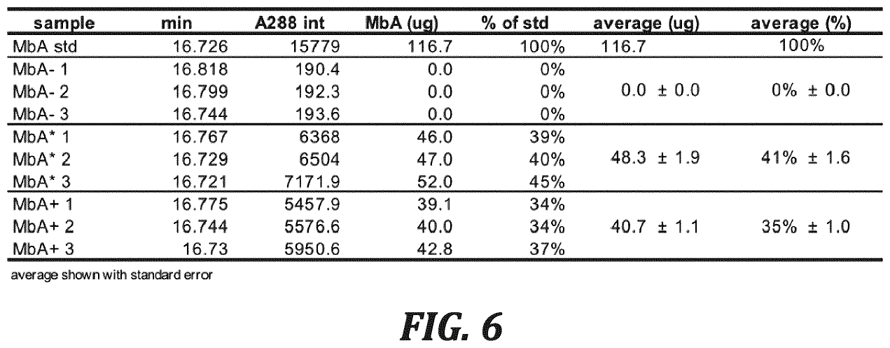

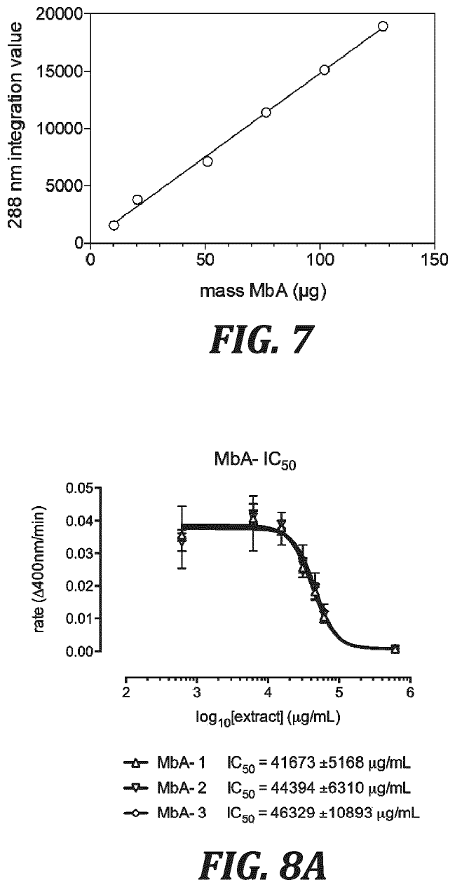

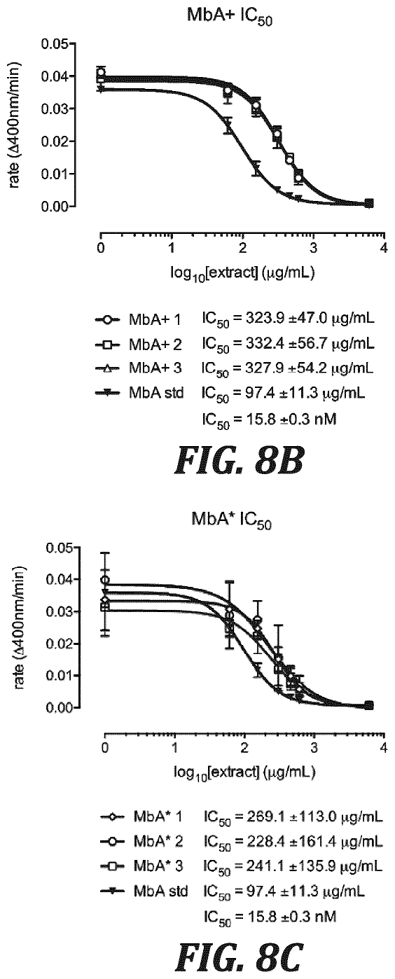

HPLC analyses for methanol extracts of MbA breads (MbA*, MbA+, and MbA−) compared to MbA standard are shown in FIGS. 5 and 6.

100 μL of each bread extract was injected on HPLC as described above. MbA was quantified by integrating the MbA on the HPLC trace to standard curve of known MbA amounts.

As used herein, the term “about” refers to ±5% of the specified value.

While illustrative embodiments have been illustrated and described, it will be appreciated that various changes can be made therein without departing from the spirit and scope of the invention.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings.

1. A cooked food product for limiting polysaccharide processing in a warm-blooded mammal, comprising montbretin A.

2. The cooked food product of claim 1, wherein the cooked food product is prepared by heating ingredients to a temperature of about 100° C. for a period of time sufficient to provide the cooked product.

3. The cooked food product of claim 1, wherein the cooked food product is a food prepared from dough.

4. The cooked food product of claim 1, wherein the cooked food product food is a baked food or a fried food.

5. The cooked food product of claim 1, wherein the cooked food product is a bread.

6. The cooked food product of claim 1, wherein the montbretin A is present in the cooked food product in an amount from about 0.1 to about 1.0 milligram per gram of the cooked food product.

7. The use of montbretin A as an ingredient in a cooked food product for human consumption for limiting polysaccharide processing in a human.

8. A method for limiting polysaccharide processing in a human subject, comprising providing an effective amount of a cooked food product of claim 1 for consumption by a human subject in need thereof.

9. The method of claim 8, wherein the effective amount of the food product is from about 0.1 gram to about 2.0 gram food product per kg human subject.

10. A method of managing postprandial glycemia in a human subject in need thereof, wherein said subject is pre-diabetic, has diabetes, or is obese, the method comprising providing an effective amount of a cooked food product of claim 1 for consumption by a human subject to manage said postprandial glycemia.

11. A method of inhibiting mammalian α-amylase in a human in need of α-amylase inhibition, the method comprising administering an effective amount of a cooked food product of claim 1 to inhibit said mammalian α-amylase.

12. The method of claim 11, wherein the α-amylase is pancreatic α-amylase or salivary α-amylase.

13. A method for treating diabetes in a human subject, comprising administering an effective amount of a cooked food product of claim 1 for consumption by a human subject for the treatment of diabetes.
